(19) United States
(12) Patent Application Publication
(10) Pub. No.: US 2024/0366414 A1
(43) Pub. Date: Nov. 7, 2024

(54) TRACTION DEVICE FOR PROMOTING GROWTH OF HUMAN PENIS

(71) Applicant: Bruce Gioia, Vancouver (CA)

(72) Inventor: Bruce Gioia, Vancouver (CA)

(21) Appl. No.: 18/777,816

(22) Filed: Jul. 19, 2024

(51) Int. Cl. A61F 5/41

(52) U.S. Cl. CPC A61F 5/41; A61F 2005/411; A61F 2005/414

(57) ABSTRACT

A penile lengthening traction device is provided. The penile lengthening traction device includes a top clamping assembly that includes a top body shell, a top body cover, a pair of top contact pads, and a top traction pad. The device includes a bottom clamping assembly that includes a bottom body shell, a bottom cover, a pair of bottom contact pads, and a bottom traction pad. The top clamping assembly and the bottom clamping assembly are operationally coupled using a pair of threaded bolts for hanging appropriate weight thereon utilizing a four-cord carabiner stabilization attachment assembly. The top clamping assembly is openable in relation to the bottom clamping assembly to accommodate a penile shaft therebetween.

### FIELD OF THE INVENTION

The present invention relates to penis extension devices, more particularly to a traction device for promoting the growth of a human penis. Specifically, the present invention relates to a clamping device that uses interchangeable traction pads with a carabiner attachment assembly that utilizes four cords to stabilize a weight under suspension to effectively stretch the penis.

### BACKGROUND

There are various methods for effective stretching of the penis with applied traction. These methods are used to correct inborn genetic defects, negative external influences, and for penis enlargement. These methods are divided into four basic types of categories.

The traditional Middle Eastern practice of “jelqing” is a technique often used for manual penis enlargement. Using this method, a semi-erect penis is stretched with a “milking” action that starts with a squeeze at the base of the penile shaft. The squeezing action is then maintained through a firm stroke that both stretches and forces blood towards the distal end of the penis. Effective jelqing stretches the penis and hyper-expands the cellular structures for maximum engorgement of the penile tissue to initiate growth. However, this method demands both physical strength and stamina which causes the practitioner's arms and hands to fatigue and tire with subsequent diminishing of the required intensity for this exercise to be effective.

Innovations mechanical in nature have been developed for penile extension. One such mechanical device features simple noose-like attachments made of cloth or leather straps secured on the penile shaft directly behind the coronal ridge of the glans. A sufficiently tightened noose acts as a secure attachment point for suspending heavy objects such as iron weights for the application of traction needed to stretch the penis.

A more sophisticated method of attachment is classified as vacuum hanging. This method uses a cup-like chamber placed over the penile glans. The cup is subsequently held in place by evacuation which creates negative pressure within the chamber for a suction grip. This type of device is relatively complex to set up as it requires interlocking components to create an airtight seal. This method frequently produces unwanted blisters that increase substantially at the higher vacuum levels generated within the chamber when the additional force of traction is applied.

Different inventors in the past have proposed penile traction devices for stretching the penis, for example, U.S. Pat. No. 6,033,374 by Miller discloses a penile traction device. The patent describes a device attached to the shaft using a foam pad wrap with a felt backing that is held in place by a hook strip. The first end of the strap is threaded through the buckle with the free end pulled to stretch back around the penile shaft to the desired tension. The attachment strap for adding weight is secured to the wrap. The weights are then hung from the strap and the force of traction is transferred to the wrap and subsequently to the penile shaft.

U.S. Pat. No. 5,599,275 by France discloses an apparatus for stretching the penis. This device is formed by the two semicircular parts joined by a hinge as required to open and close the apparatus for putting on and taking off the device. In the closed position, a complete circle is formed around the penis shaft. A groove around the outside perimeter of the two halves is recessed to accept an elastic band for securing the clamp in the closed position. The device weighs between 10 to 16 ounces. The apparatus is not designed to accept additional weight for increased traction. The inner diameter of the two parts is of a fixed circumference so the user must choose the correct size for the proper fit.

U.S. Pat. No. 7,377,896 by Dykers Jr. discloses an inflatable penis ring. The device comprises an annular ring with an array of inflatable balloons evenly spaced around the internal circumference. The penis is inserted through the internal diameter of balloons to the preferred position on the shaft. An external manual pump is used to inflate or deflate the balloons for adjusting the degree of compression to effectively secure the annular ring on the shaft of the penis. The ring facilitates the attachment of various medical apparatus including the suspension of weight for applied traction.

BibHanger™ is a penis enlargement device available in the market. This penis enlargement device utilizes a standard “off the shelf” door hinge to act as a fulcrum between the two sides of the clamping mechanism. A long thin threaded bolt utilizes a wing nut for tightening the clamp on the shaft. The clamping surface includes a series of ridges with a dimpled surface for added traction. These ridges are angled off-center for clamping onto either side of the penile shaft parallel to the angled slope of the glans. A single length of nylon webbing is bolted to either side of the clamp for attaching an “S” hook to suspend weight.

Malehanger™ is another penis stretcher device available in the market. This penis stretcher device relies heavily on its predecessor the “BIB Hanger”. The Malehanger has been adapted for manufacture with plastic injection parts. This device utilizes the same “off the shelf” door hinge as the pivot point between the two halves of the clamp. This device also relies on the same undersized threaded bolt with an inefficient wing nut to screw the clamp down tightly on the shaft. However, with repeated use, the threads of the undersized bolt inevitably strip and therefore require routine replacement. The clamping surface includes the same angled ridges with the dimpled surface as the original “BibHanger” as well as the same single strap of webbing bolted to the two halves of the device for attaching the “S” hook to suspend weight.

Total Man™ is another penis enlargement clamping device available in the market with a metal dowel acting as a pivot point. However, the same wing nuts are used to screw the clamp down for a sufficiently tight grip. This apparatus utilizes two sets of smooth interchangeable “spacer plates” of different thicknesses that are designed to clamp on smaller and larger diameter penis shafts. A single length of paracord is used with concealed knots as a method of attachment on either side of the clamp. The paracord is used for attaching an “S” shaped hook to suspend weight for applied traction.

Although different solutions for enlargement of the penis have existed, there still exists a compression clamp traction device for penis enlargement or promoting the growth of the human penis. The present invention utilizes a clamping device to create a compression grip on the penile shaft directly behind the glans. The practitioner/user can choose to use a variety of interchangeable traction pads to find their preferred fit and gripping style on the penile shaft. The compression clamp, according to the present invention, also includes a secondary assembly connected by four cords to provide greater stability of the carabiner attachment point that is required for suspending weight.

### BRIEF SUMMARY

After careful consideration of the prior art, it is readily apparent that the existing design approaches to date fail to realize optimal performance in this category of device. Therefore, a more efficient clamping device for penis extension is required to improve the user experience.

The current invention includes several novel features that effectively resolve the existing problems found in the prior art, which are listed as follows: 1. Facilitate greater ease of use to put on, wear, and remove the device. 2. Provide interchangeable traction pads for individual grip preferences. 3. Utilize four cords to stabilize the weight under suspension. 4. Include a suspension assembly for a balanced carabiner attachment point.

A prerequisite of an optimized clamping device for stretching the penis requires that it is convenient to set up, put on, and wear, while at the same time, easy to take off for removal. The proposed invention achieves this using heavy gauge threaded bolts with large easy to manipulate thumb nuts to facilitate effortless manual opening, setting, and closing of the clamp for a powerful compression grip.

The two sides of the clamp are structured with internal ribs for a streamlined exterior that does not twist and distort under pressure. The internal ribs also provide concealed fulcrum pin housings which provide the full range of motion for the threaded bolts with powerfully levered thumb nuts to efficiently adjust the clamping device.

The interchangeable traction pads of the device provide several tread patterns for an efficient and comfortable grip of the penile shaft. The pad selection includes a variety of channels that allow preferential adjustments for different penis shaft diameters and grip characteristics.

The sleek and aesthetically designed body of the clamping device includes four holes to receive the four suspension cords that connect to the stabilization platform of the carabiner attachment assembly. The four cords facilitate stable distribution of the weight under suspension. The four-cord method effectively prevents the device from leaning off center as it does when suspended by a single cord or length of cord or webbing attached to either side of the device for mounting the “S” hook.

To attain these advantages, embodiments of the present invention disclose a penile lengthening traction device is provided. The penile lengthening traction device includes a top clamping assembly that includes a top body shell, a top body cover, a pair of top contact pads, and a top traction pad. The device includes a bottom clamping assembly that includes a bottom body shell, a bottom cover, a pair of bottom contact pads, and a bottom traction pad. The top clamping assembly and the bottom clamping assembly are operationally coupled using a pair of threaded bolts for hanging appropriate weight thereon utilizing a four-cord carabiner attachment assembly. The top clamping assembly is openable in relation to the bottom clamping assembly to accommodate a penile shaft therebetween.

These and other features and advantages of the present invention will become apparent from the detailed description below, in light of the accompanying drawings.

### BRIEF DESCRIPTION OF THE ACCOMPANYING DRAWINGS

The novel features which are believed to be characteristic of the present invention, as to its structure, organization, use, and method of operation, together with further objectives and advantages thereof, will be better understood from the following drawings in which a presently preferred embodiment of the invention will now be illustrated by way of example. It is expressly understood, however, that the drawings are for illustration and description only and are not intended as a definition of the limits of the invention. Embodiments of this invention will now be described by way of example in association with the accompanying drawings in which:

FIG. 1 illustrates an exploded view of a penile lengthening traction device assembly, according to an exemplary embodiment of the present invention.

FIG. 2 illustrates an exploded view of a bottom clamping assembly of the penile lengthening traction device assembly of FIG. 1.

FIG. 3 illustrates a partially exploded view of the bottom clamping assembly of FIG. 2 along with two contact pads and an interchangeable traction pad.

FIG. 4 illustrates a top clamping assembly and a bottom clamping assembly with attached traction pads before being screwed down and clamped together with thumb nuts.

FIG. 5 illustrates an assembled view of the penile lengthening traction device assembly with the top and bottom clamping assemblies assembled using thumb nuts.

FIG. 6 illustrates the top and bottom clamping assemblies of the penile lengthening traction device assembly in a half open state with one thumb nut removed for visual clarity.

FIG. 7 illustrates the assembled view of the bottom clamping assembly of the penile lengthening traction device assembly with attached contact pads and unattached traction pads.

FIG. 8 illustrates four sets of interchangeable traction pads for use with the penile lengthening traction device assembly, according to an exemplary embodiment.

FIG. 9 illustrates an exemplary method of attaching the suspension cords to the bottom body shell of the bottom clamping assembly.

FIG. 10 illustrates an exploded view of a four-cord carabiner attachment assembly, according to an embodiment of the present invention.

FIG. 11 illustrates an assembled view of the four-cord carabiner attachment assembly of FIG. 10.

FIG. 12 illustrates a perspective view of the penile lengthening traction device assembly together with the four-cord carabiner attachment assembly of FIG. 11.

### DETAILED DESCRIPTION

As used in the specification and claims, the singular forms “a”, “an”, and “the” may also include plural references. For example, the term “an article” may include a plurality of articles. Those with ordinary skill in the art will appreciate that the elements in the Figures are illustrated for simplicity and clarity and are not necessarily drawn to scale. For example, the dimensions of some of the elements in the figures may be exaggerated, relative to other elements, to improve the understanding of the present invention. There may be additional components described in the foregoing application that are not depicted in one of the described drawings. In the event such a component is described, but not depicted in a drawing, the absence of such a drawing should not be considered as an omission of such design from the specification.

Before describing the present invention in detail, it should be observed that the present invention utilizes a combination of components or set-ups, which constitutes a compression clamp traction device for penis lengthening or a traction device for promoting the growth of a human penis. Accordingly, the components have been represented, showing only specific details that are pertinent for an understanding of the present invention so as not to obscure the disclosure with details that will be readily apparent to those with ordinary skill in the art having the benefit of the description herein. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting but rather to provide an understandable description of the invention.

References to “one embodiment”, “an embodiment”, “another embodiment”, “yet another embodiment”, “one example”, “an example”, “another example”, “yet another example”, and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in an embodiment” does not necessarily refer to the same embodiment.

The words “comprising,” “having,” “containing,” and “including,” and other forms thereof, are intended to be equivalent in meaning and be open ended in that an item or items following any one of these words is not meant to be an exhaustive listing of such item or items or meant to be limited to only the listed item or items. Unless stated otherwise, terms such as “first” and “second” are used to arbitrarily distinguish between the elements. Thus, these terms are not necessarily intended to indicate temporal or other prioritization of such elements. While various exemplary embodiments of the disclosed system and method have been described above it should be understood that they have been presented for purposes of example only, not limitations. It is not exhaustive and does not limit the invention to the precise form disclosed. Modifications and variations are possible considering the above teachings or may be acquired from practicing the invention, without departing from the breadth or scope.

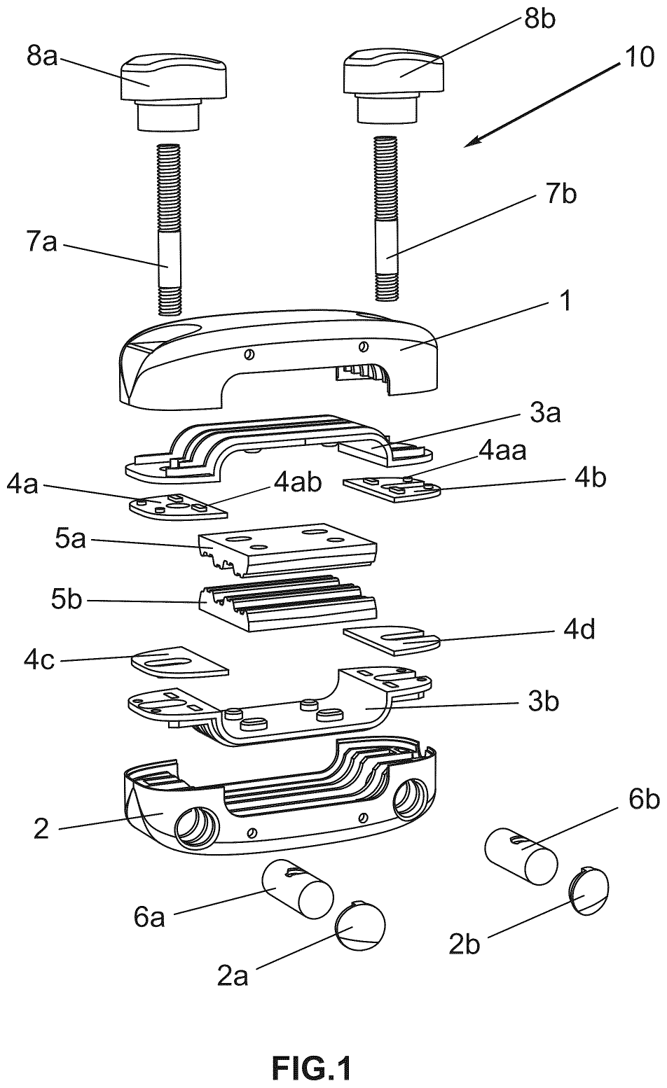

Referring to FIG. 1, an exploded view of a penile lengthening traction device assembly 10, according to an exemplary embodiment of the present invention is shown. As seen, the device 10 includes a top clamping assembly comprising a top body shell 1, a top body cover 3a, a pair of top contact pads (4a, 4b), and a top traction pad 5a. The device 10 further includes a bottom clamping assembly comprising a bottom body shell 2, a pair of caps (2a, 2b) configured to be used with the bottom body shell 2, a bottom cover 3b, a pair of bottom contact pads (4c 4d), a bottom traction pad 5b, a pair of fulcrum pins (6a 6b) that slides into the bottom body shell 2 and enclosed using the caps (2a,2b). The device 10 further comprises a pair of threaded bolts (7a 7b), and a pair of thumb nuts (8a, 8b) to operationally couple the top and bottom clamping assemblies together and operationally manipulate or open or close the top clamping assembly in relation to the bottom clamping assembly, thereby allowing to accommodate penile shaft therebetween for hanging appropriate weight onto the device 10 for required traction. The top body shell 1, the bottom body shell 2, the top body cover 3a, the bottom body cover 3b, the caps (2a, 2b), and the fulcrum pins (6a 6b), are all rigid structures formed of rigid or hard material selected from plastic, ABS, metal, wood or any other suitable material known in the art.

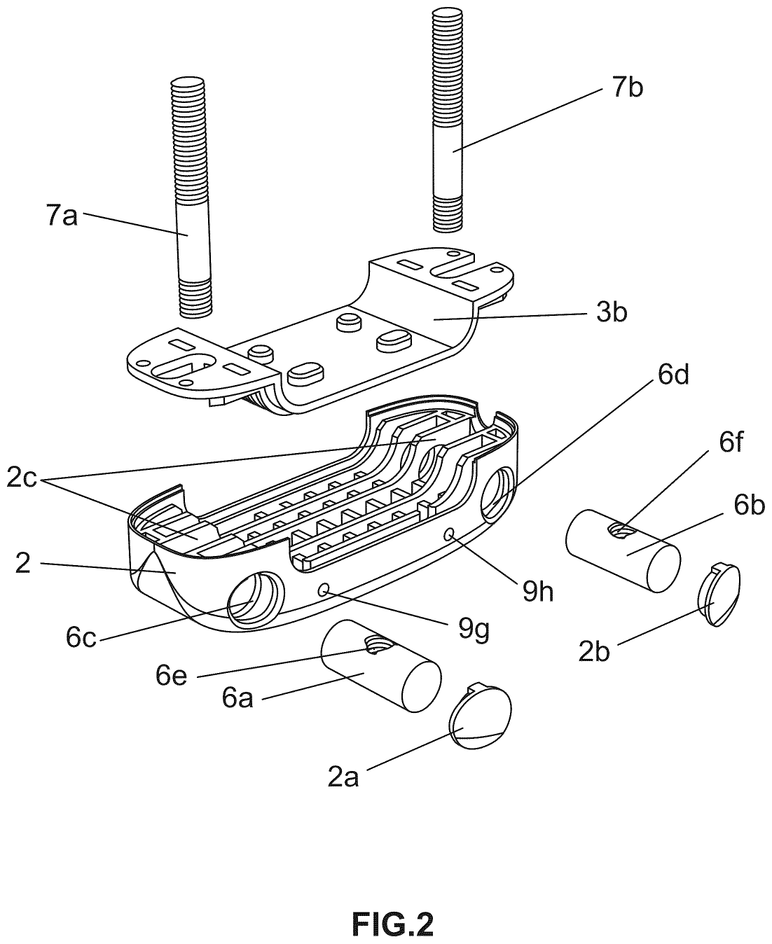

Referring to FIG. 2, an exploded view of a bottom clamping assembly of the penile lengthening traction device is shown. As seen, the bottom clamping assembly includes the bottom body shell 2 substantially U-shaped having internal ribs 2c to provide not only strength and rigidity to the bottom body shell 2 but also to provide a pair of negative spaces (6c 6d) for housing the fulcrum pins (6a 6b) therein. These negative spaces (6c 6d) after receiving the fulcrum pins (6a 6b) are closed using the caps (2a,2b). As seen, the fulcrum pin 6a comprises a female threaded cavity 6e and the fulcrum pin 6b comprises a female threaded cavity 6f for receiving the threaded bolt 7a and the threaded bolt 7b respectively there inside. The fulcrum pins (6a,6b) allow the full range of motion for the threaded bolts (7a,7b) that are required for clamping the top body shell 1 (not shown) down tight or open and separate from the bottom body shell 2. A set of two holes (9g, 9h) are provided in the bottom body shell 2 for receiving suspension cords there inside (not shown). Although not seen, the top body shell 1 also includes internal ribs 2c to provide strength and rigidity to the top body shell 1.

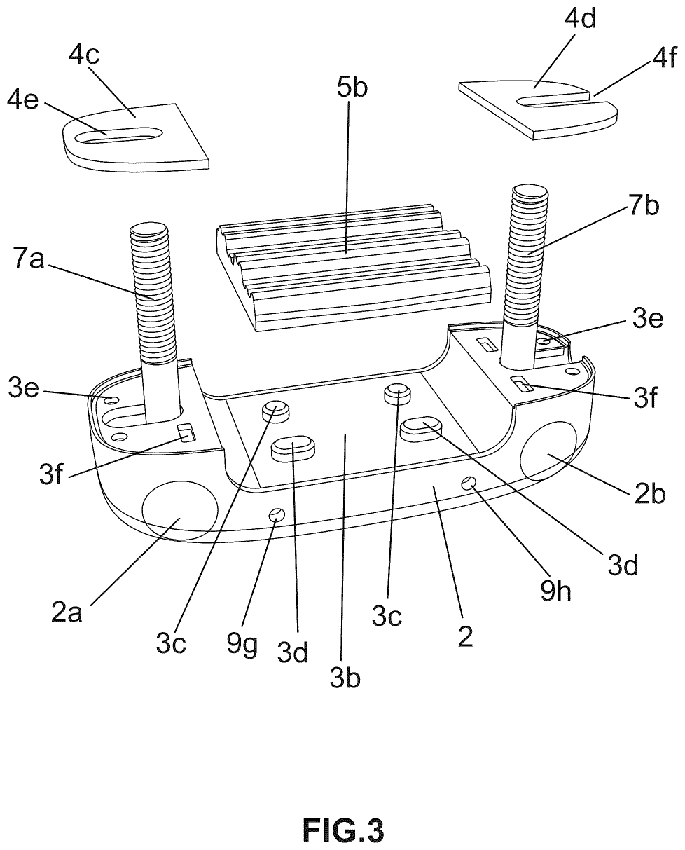

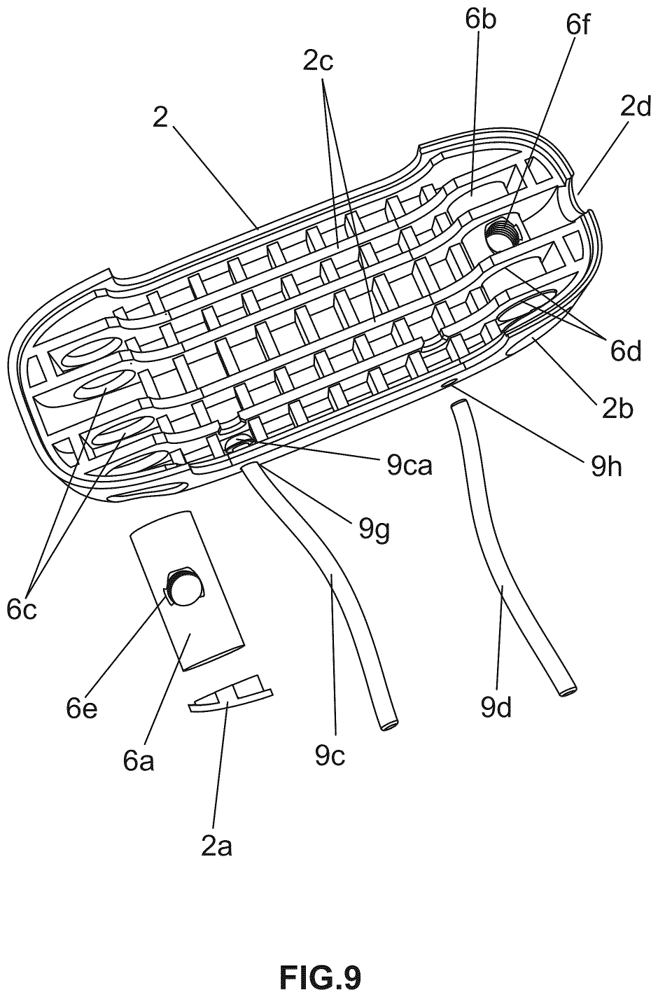

Referring to FIG. 3, a partially exploded view of the bottom clamping assembly of FIG. 2 along with two contact pads and an interchangeable traction pad is shown. FIG. 3 thus shows additional components associated with the bottom clamping assembly which are not shown in FIG. 2. As seen in FIG. 3, the bottom clamping assembly includes the bottom body shell 2, the body caps (2a, 2b), the bottom cover 3b adapted to cover the internal ribs 2c provided on the body shell 2, the threaded bolts (7a, 7b), the bottom contact pads (4c and 4d) configured underside at two ends of the bottom body shell 2 over the bottom cover 3b, and an interchangeable traction pad 5b. The bottom cover 3b is also substantially U-shaped (like bottom body shell 2) configurable underside of the bottom shell 2 to cover the internal ribs 2c. The body cover 3b includes a plurality of protrusions, namely two circular protrusions 3c and two rounded rectangular protrusions 3d in the form of external male-shaped nibs for attachment to the interchangeable traction pad 5b in the correct orientation. The underside of the interchangeable traction pad 5b comprises female grooves (not seen) complementing to the male shaped nibs (3c,3d). The body cover 3b further includes a plurality of female spaces, namely four similarly shaped internal circular spaces 3e, two each on opposing sides, and four internal rounded rectangular female spaces 3f, two on each side to align and receive the bottom contact pads (4c, 4d) configurable on the body cover 3b. The female spaces 3e,3f are identically shaped to match corresponding male-shaped nibs or protrusions (4aa,4ab) (see FIG. 1) present on the underside of the bottom contact pads (4c, 4d). Although the protrusions (4aa,4ab) are only seen on the top contact pads (4a,4b), it should be appreciated that similar protrusion arrangements exist underside the bottom contact pads (4c,4d). As seen, the contact pad 4c includes a closed slot 4e for limited rotation of threaded bolt 7a, and the contact pad 4d includes an open-ended slot 4f for threaded bolt 7b to swing wide open. The two holes 9g and 9h located on the body shell 2 receive the suspension cords 9c and 9d respectively (seen in FIG. 9).

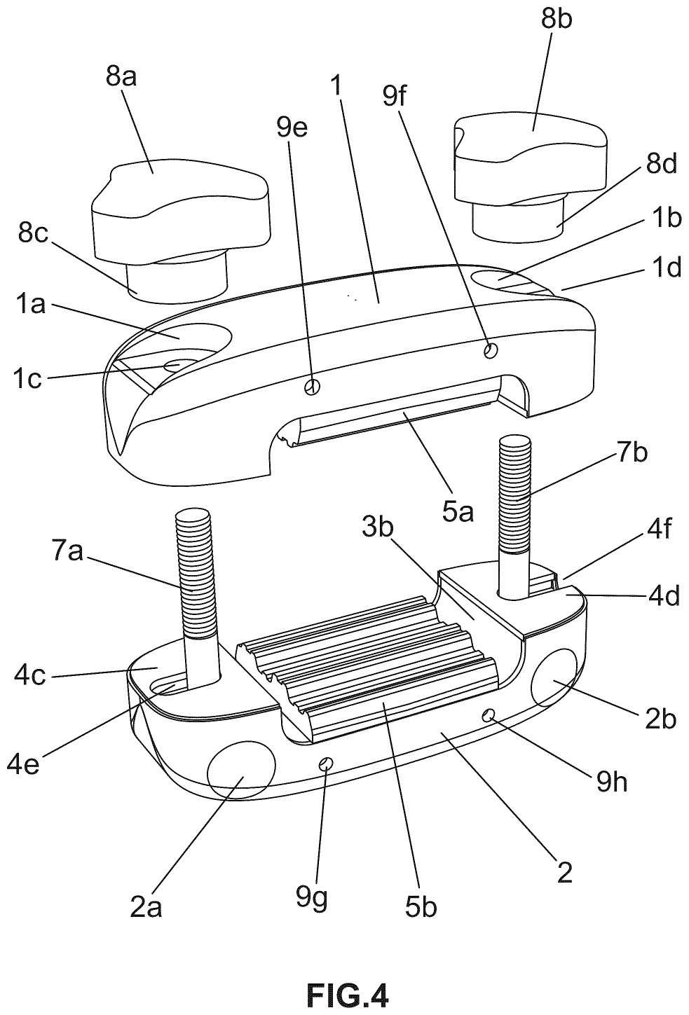

Referring to FIG. 4, a top clamping assembly and a bottom clamping assembly with attached traction pads is shown before being screwed down and clamped together using thumb nuts. As seen, the assembled top clamping assembly and the assembled bottom clamping assembly are shown with unscrewed/detached thumb nuts (8a, 8b) having bases (8c, 8d) respectively. The top body shell 1 comprises a pair of receptacles (1a,1b) configured for receiving the bases (8c, 8d) of the thumb nuts (8a,8b). The top body shell 1 includes a bolt hole 1c and another slotted bolt hole 1d configured within the receptacles (1a,1b) respectively. In FIG. 4, the top body cover (not seen), and the top contact pads (not seen). However, a detachable traction pad 5a is shown attached. Further as seen, the detached fully assembled bottom body shell 2 is shown to include the caps (2a, 2b), the bottom body cover 3b with detachable traction pad 5b is shown attached, the contact pad 4c is shown to include the closed slot 4e for limited rotation of the threaded bolt 7a while the contact pad 4d is shown to include an open-ended slot 4f that allows the threaded bolt 7b to swing wide open. The top body shell 1 includes a plurality of holes, namely two holes 9e and 9f to receive the suspension cords (see FIG. 9) and the bottom body shell 2 includes a plurality of holes, namely two holes 9g and 9h to receive the suspension cords (see FIG. 9).

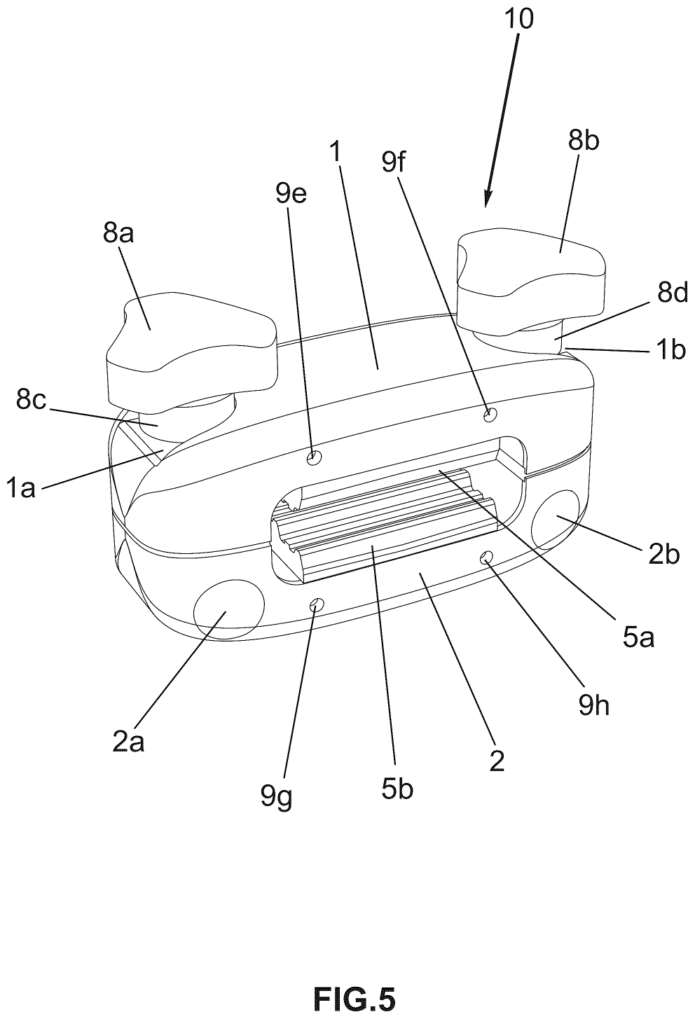

Referring to FIG. 5, an assembled view of the penile lengthening traction device assembly 10 with the top and bottom clamping assemblies assembled using thumb nuts is shown. In assembly, the screwed top and bottom clamping assemblies are clamped tight using the thumb nuts (8a, 8b) having the bases (8c, 8d). The bases (8c,8d) of the nuts (8a,8b) are aligned into the receptacles (1a, 1b) provided on the top body shell 1. Particularly, the nuts (8a,8b) are screwed over the threaded bolts (7a,7b) that engage the bottom body shell 2 with the top body shell 1. Specifically, the threaded bolts (7a,7b) which are screwed onto the fulcrum pins (6a,6b) of the bottom body shell 2 pass through the closed slot 4e, and the open-ended slot 4f respectively located on the contact pads (4c,4d) of the bottom shell 2 and received within the bolt holes (1c,1d) located on the receptacles (1a,1b) of the top shell 1 to finally protrude out of the holes (1c,1d) for the nuts (8a,8b) to screw over them. The detachable traction pads 5a, 5b are attached to the top body shell 1 and the bottom body shell 2 respectively. The bottom shell 2 includes the caps (2a, 2b) for closure of the negative spaces (6c 6d) housing the fulcrum pins (6a 6b). The holes (9e, 9f) of the top body shell 1 receive the suspension cords (9a, 9b) respectively, and the holes (9g, 9h) of the bottom body shell 2 receive the suspension cords (9c, 9d) respectively (see FIG. 9).

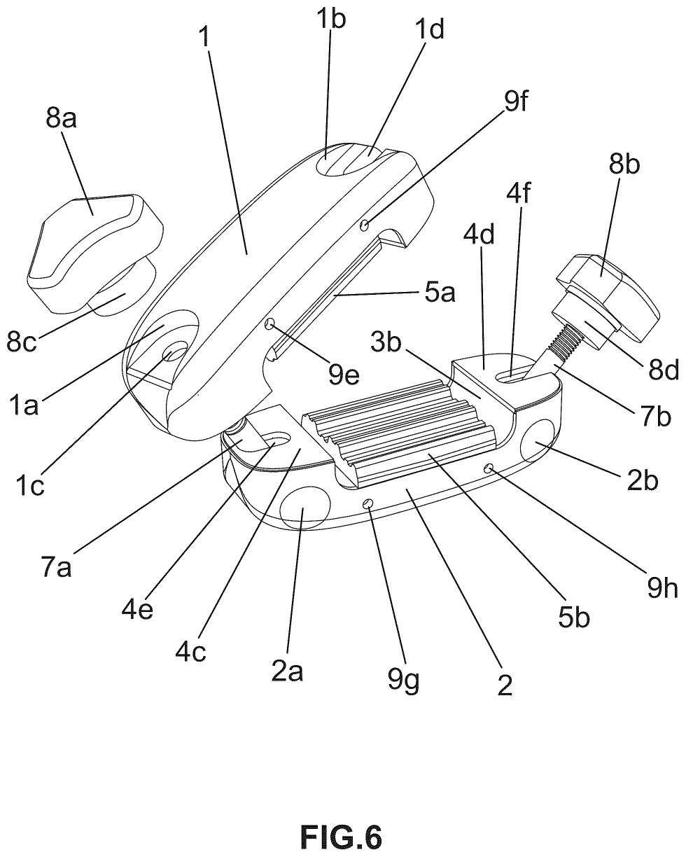

Referring to FIG. 6, the top and bottom clamping assemblies of the penile lengthening traction device assembly are shown partially opened state (half open) with one thumb nut removed for visual clarity. In an embodiment, as seen, the assembled perspective view of the device 10 is shown with the thumb nut 8a unscrewed and detached and the thumb nut 8b swung wide open to showcase a clear view of the clamping mechanism. In the fully open position, the top body shell 1 with the thumb nut alignment receptacles (1a, 1b) is seen for positioning the bases 8c and 8d of the thumb nuts 8a,8b. The top body shell 1 includes the bolt hole 1c and slotted bolt hole 1d to both guide and limit the range of motion of the threaded bolts 7a and 7b. The top body shell 1 has the interchangeable top traction pad 5a attached thereto, and the bottom body shell 2 has the interchangeable bottom traction pad 5b. The bottom shell 2 further includes caps 2a and 2b, and the bottom body cover 3b, with the contact pad 4c that includes a closed slot 4e for limited rotation of threaded bolt 7a and the contact pad 4d that includes an open-ended slot 4f which allows the threaded bolt 7b to swing wide open. The two holes (9e, 9f) in the top body shell 1 are configured to receive the suspension cords (9a, 9b) respectively, and the two holes (9g, 9h) in the bottom body shell 2 are configured to receive the suspension cord (9c,9d) respectively (see FIG. 9).

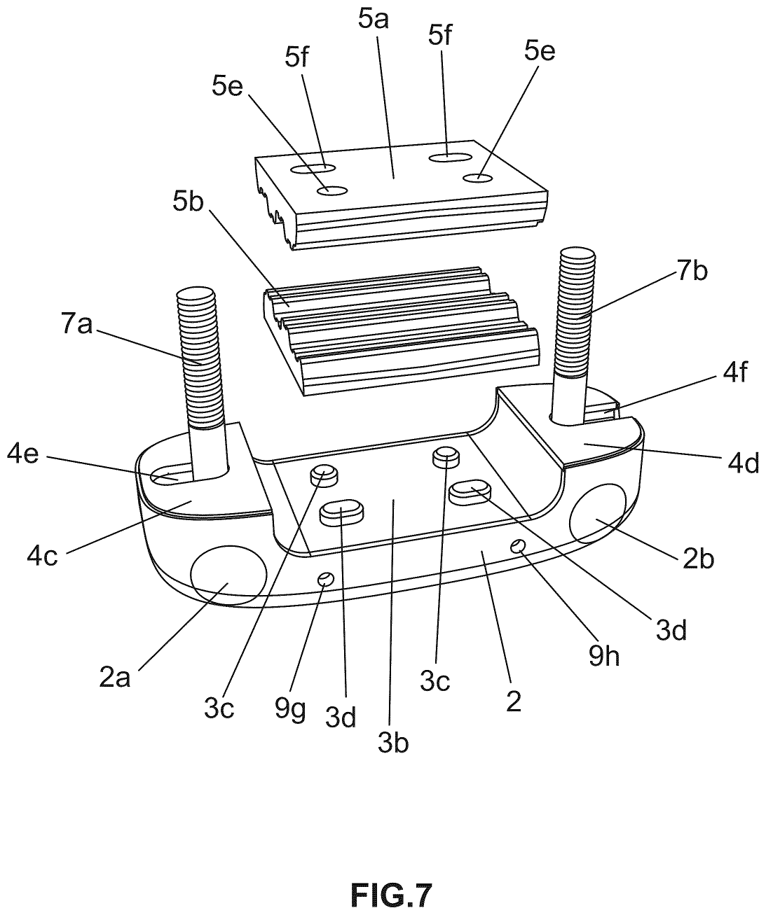

Referring to FIG. 7, the assembled view of the bottom clamping assembly of the penile lengthening traction device assembly with attached contact pads and unattached traction pads is shown. As seen, the bottom body shell 2, the caps (2a, 2b), the bottom body cover 3b with attached/bonded contact pads (4c, 4d), the threaded bolt 7a that passes through the closed slot 4e for limited rotation, and the threaded bolt 7b passing through the open-ended slot 4f to allow the threaded bolt 7b to swing wide open are seen. Further, the bottom body cover 3b is shown to include protrusions, namely two circular protrusions 3c and two rounded rectangular protrusions 3d in the form of external male-shaped nibs for attachment to the interchangeable traction pad 5b in the correct orientation. Although not seen, the top body shell also includes the top body cover 3a includes protrusions, namely two circular protrusions and two rounded rectangular protrusions in the form of external male-shaped nibs for attachment to the interchangeable traction pad 5a in the correct orientation. FIG. 7 shows the top traction pad 5a with negative female spaces in the circular shape 5e and rounded rectangular shape 5f shapes which attach to the top body cover 3a (not seen). Similarly, spaces are configured underside the bottom traction pad 5b to enable the traction pad 5b to removably attach over the protrusions 3c,3d (the circular shaped spaces 5e align with the circular protrusions 3c, whereas the rectangular shaped spaces 5f align with the rectangular shaped protrusions 3d). The external male-shaped nibs or protrusions (3c, 3d) of the bottom body cover 3b provide the required friction fit for the bottom traction pad 5b and allow it to be mounted in the correct orientation. Likewise, the male-shaped nibs or protrusions (3c, 3d) of the top body cover 3a provide the required friction fit for the top traction pad 5b and allow it to be mounted in the correct orientation on the top cover 3a. The two holes (9g, 9h) in the lower hard body shell 2 receive the suspension cord (9c, 9d) respectively (see FIG. 9).

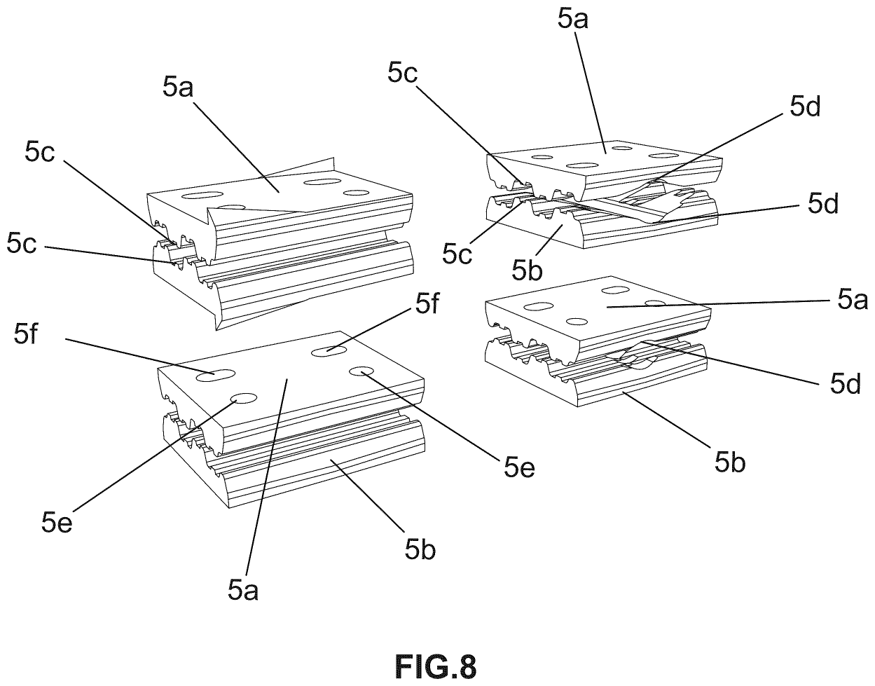

Referring to FIG. 8, four sets of interchangeable traction pads for use with the penile lengthening traction device assembly 10, according to an exemplary embodiment are shown. As seen, four sets of interchangeable traction pads are shown paired together. The top traction pad 5a and the bottom traction pad 5b have identical tread patterns 5c so that the pairs interlock squarely when flipped over to meet face-to-face. In some embodiments, the top traction pads 5a and bottom traction pads 5b may include the two centre channels 5d running through the middle of the pads 5a,5b. The interchangeable pads 5a,5b are easily swapped in and out of the device 10 according to the users' preference. The traction pads 5a,5b are attached to the top body covers 3a and the bottom body cover 3b of the assembled device (not shown) by means of the two internal female circular spaces 5e and two internal female rounded rectangular spaces 5f for a friction fit on the top and bottom body covers (see FIG. 7). This is achieved by insertion of the two corresponding external male shaped nibs or protrusions 3c, 3d which are part of the top body over 3a and the bottom body covers 3b (see FIG. 7) for the required friction fit.

FIG. 9 illustrates an exemplary method of attaching the suspension cords to the bottom body shell of the bottom clamping assembly. FIG. 9 specifically shows the bottom body shell 2, the cap 2a (exploded) with the cap 2b assembled in place enclosing the fulcrum pin (not seen). The exposed array of internal horizontal and vertical ribs 2c configured on the bottom body shell 2 provide additional strength and rigidity to the bottom shell 2. The two negative spaces (6c, 6d) are present in the bottom body shell 2 required for housing the fulcrum pins 6a (exploded) and 6b (attached enclosing the fulcrum pin) for rotation of the threaded bolts (7a, 7b) (not shown) The two threaded bolts are screwed into the threaded holes (6e, 6f) of the two fulcrum pins (6a 6b). The bottom body shell 2 also includes a slotted opening 2d for the threaded bolt 7b (not shown) to swing wide open. The bottom body shell 2 also includes two holes 9g and 9h for inserting two of four suspension cords (9c, 9d). After insertion of the suspension cord 9c through the hole 9g of the body shell 2, the cut end is flame melted to form a globular mushroom-shaped head 9ca to secure itself within the body shell 2. The suspension cord 9d is not shown inserted through hole 9h of the body shell 2 and is therefore shown without the melted globular mushroom shaped head that is required for securing itself within the body shell 2.

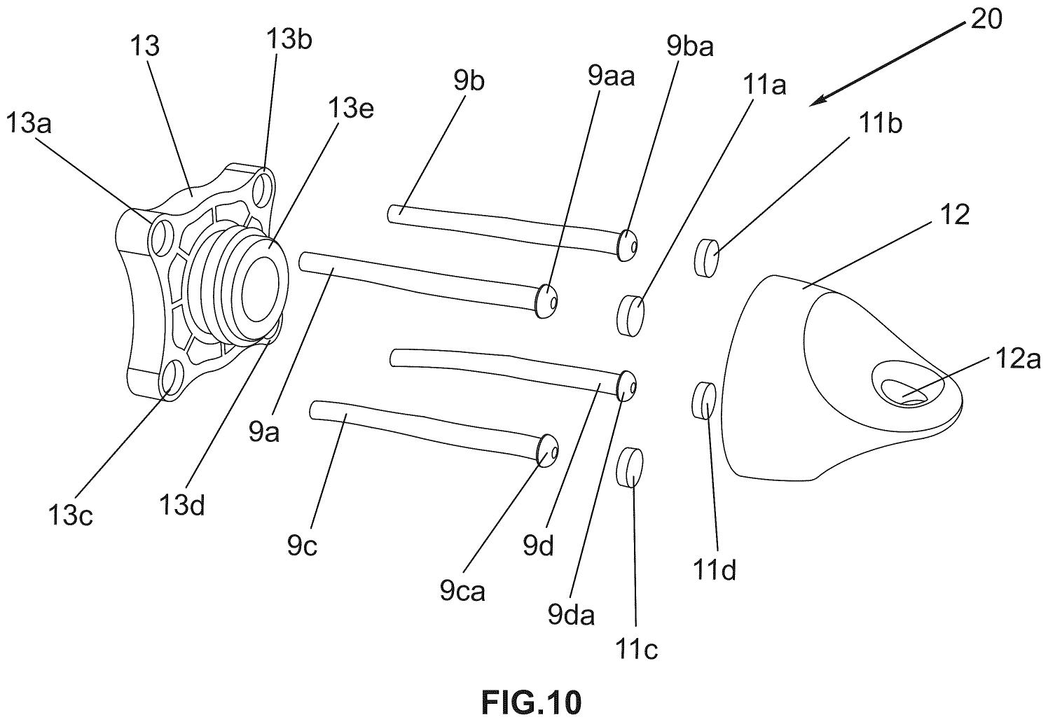

Referring to FIG. 10, an exploded view of a four-cord carabiner attachment assembly, according to an embodiment of the present invention. As seen, the carabiner attachment assembly 20 includes four suspension cords 9a, 9b, 9c, and 9d, each with melted globular mushroom-shaped heads 9aa, 9ba, 9ca, 9da respectively. The carabiner attachment assembly 20 includes a stabilization platform 13 with four suspension cord receptacle holes 13a, 13b, 13c, 13d with threaded screw attachment knob 13e, four concealment caps 11a, 11b, 11c, 11d, and a body 12 of the carabiner attachment assembly 20 with a carabiner attachment hole 12a.

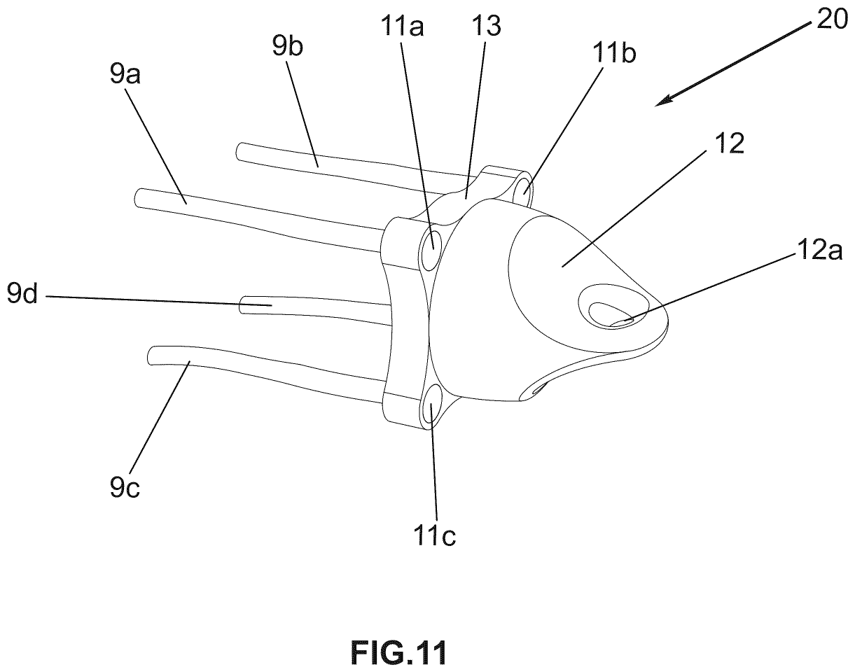

Referring to FIG. 11, an assembled view of the four-cord carabiner attachment assembly of FIG. 10. As seen, the suspension cords 9a, 9b, 9c, and 9d are threaded through the stabilization platform 13, specifically through the receptacle holes 13a, 13b, 13c, 13d (not seen) with corresponding concealment caps 11a, 11b, 11c, 11d. The body 12 of the carabiner attachment assembly 20 is screwed into place over the threaded screw attachment knob 13e (not seen) of the stabilization platform 13. The body 12 of the carabiner attachment assembly 20 comprises the carabiner attachment hole 12a for attaching an external carabiner for suspending a desired weight.

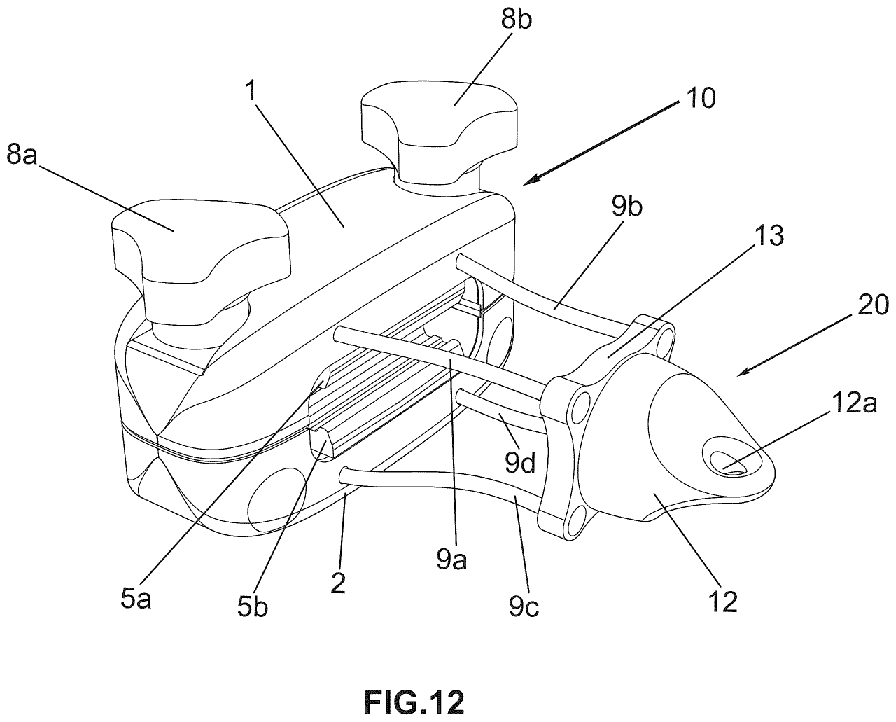

Referring to FIG. 12, a perspective view of the penile lengthening traction device assembly 10 together with the four-cord carabiner attachment assembly 20 of FIG. 11 is shown. FIG. 12 particularly depicts a fully assembled view of the device 10 with the top body shell 1, the bottom body shell 2, the detachable top and bottom traction pad (5a, 5b), the thumb nuts (8a, 8b), the four suspension cords 9a, 9b, 9c, 9d, the stabilization platform 13, and the body 12 of the carabiner attachment assembly 20 attached to the stabilization platform 13 with the hole 12a for attaching a carabiner for suspending weight. Inclusion of four holes 13a, 13b, 13c, 13d to receive the four suspension cords that connect to the stabilization platform 13 of the carabiner attachment assembly 20. The four cords 9a, 9b, 9c, and 9d facilitate stable distribution of the weight under suspension. The four-cord method effectively prevents the device 10 from leaning off center as it does when suspended by a single cord or length of cord or webbing attached to either side of the device for mounting the “S” hook or carabiner hook.

The various components of the embodiments of the penile lengthening traction device of the present invention are similar and interchangeable. It is obvious to the one skilled in the art that the various components and parts of the penile lengthening traction device of one embodiment of the present invention could be considered for other embodiments with little or no variation. Further, the material used for the construction of the penile lengthening traction device and various components may include any material including but not limited to metal, rigid material, wooden material, hard plastic, ABS, and so on.

It should be understood according to the preceding description of the present invention that the same is susceptible to changes, modifications, and adaptations, and that the said changes, modifications, and adaptations fall within the scope of the appended claims.

What is claimed is:

1. A penile lengthening traction device (10), comprising:
a top clamping assembly comprising
a top body shell (1),
a top body cover (3a),
a pair of top contact pads (4a, 4b), and
a top traction pad (5a);
a bottom clamping assembly comprising
a bottom body shell (2),
a bottom cover (3b),
a pair of bottom contact pads (4c 4d), and
a bottom traction pad (5b);
wherein, the top clamping assembly and the bottom clamping assembly are operationally coupled using a first threaded bolt (7a), and a second threaded bolt (7b) for hanging appropriate weight thereon; and
wherein the top clamping assembly is openable in relation to the bottom clamping assembly to accommodate a penile shaft therebetween.

2. The penile lengthening traction device (10) of claim 1 further comprises a pair of fulcrum pins (6a 6b) disposed into the bottom body shell (2).

3. The penile lengthening traction device (10) of claim 1, wherein the pair of fulcrum pins (6a 6b) disposed into the bottom body shell (2) are enclosed using a pair of caps (2a,2b).

4. The penile lengthening traction device (10) of claim 1, wherein the top body shell (1), and the bottom body shell (2) comprises a plurality of internal ribs (2c) that provide added strength and rigidity.

5. The penile lengthening traction device (10) of claim 1, wherein the bottom body shell (2) comprises a pair of negative spaces (6c 6d) for housing the fulcrum pins (6a 6b) therein.

6. The penile lengthening traction device (10) of claim 2, wherein the pair of fulcrum pins (6a 6b) disposed into the bottom body shell (2) allows the threaded bolts (7a, 7b) to at least perform:
clamp the top clamping assembly, and the bottom clamping assembly tight holding the penile shaft therebetween,
open the top clamping assembly from the bottom clamping assembly for insertion or removal of the penile shaft therebetween, and
separate the top clamping assembly from the bottom clamping assembly completely.

7. The penile lengthening traction device (10) of claim 1, wherein the top body cover (3a) of the top clamping assembly and the bottom cover (3b) of the bottom clamping assembly are adapted to cover the plurality of internal ribs (2c) on the top body shell (1) of the top clamping assembly and the bottom body shell (2) of the bottom clamping assembly.

8. The penile lengthening traction device (10) of claim 1, wherein the top body cover (3a), and the bottom body cover (3b) comprises a plurality of first protrusions (3c,3d) in the form of external male-shaped nibs for detachably attaching the top traction pad (5a), and the bottom traction pad (5b) in correct orientation on the top body cover (3a), and the bottom body cover (3b) respectively.

9. The penile lengthening traction device (10) of claim 1, wherein the top body cover (3a), and the bottom body cover (3b) further comprises a plurality of second female spaces (3e,3f) for detachably attaching the top contact pads (4a, 4b), and the bottom contact pads (4c, 4d) in correct orientation on the top body cover (3a), and the bottom body cover (3b) respectively.

10. The penile lengthening traction device (10) of claim 7, wherein the top traction pad (5a), and the bottom traction pad (5b) comprises a plurality of first female spaces (5e,5f) that engages with the plurality of first protrusions (3c,3d) for detachably attaching the top traction pad (5a), and the bottom traction pad (5b) on the top body cover (3a), and the bottom body cover (3b) respectively.

11. The penile lengthening traction device (10) of claim 8, wherein the top contact pads (4a, 4b), and the bottom contact pads (4c, 4d) comprises a plurality of second protrusions (4aa,4ab) for detachably attaching the top contact pads (4a, 4b), and the bottom contact pads (4c, 4d) to the top body cover (3a), and the bottom body cover (3b) respectively.

12. The penile lengthening traction device (10) of claim 1, wherein one of the pair of top contact pads (4a, 4b) and the pair of bottom contact pads (4c 4d) include a closed slot (4e) for limited rotation of the first threaded bolt (7a) and one of the pair of top contact pads (4a, 4b) and the pair of bottom contact pads (4c 4d) include an open-ended slot (4f) for the second threaded bolt (7b) to swing wide open.

13. The penile lengthening traction device (10) of claim 1, wherein the top body shell (1) comprises a first set of holes (9e, 9f) and the bottom body shell (2) comprises a second set of holes (9g,9h) for receiving a first set of suspension cords (9a, 9b), and a second set of suspension cords (9c, 9d) for attachment to a four-cord carabiner attachment assembly (20) having a carabiner attachment hole (12a) for suspending a desired weight.

14. The penile lengthening traction device (10) of claim 12, wherein the four-cord carabiner attachment assembly (20) comprises a stabilization platform (13) with a plurality of suspension cord receptacle holes (13a-13d), a threaded screw attachment knob (13e), and a body (12) with the carabiner attachment hole (12a).

15. The penile lengthening traction device (10) of claim 12, wherein
the first set of suspension cords (9a, 9b) connects the top body shell (1) with the stabilization platform (13) by passing through a set of suspension cord receptacle holes (13a, 13b) selected from the plurality of suspension cord receptacle holes (13a-13d); and
the second set of suspension cords (9c, 9d) connects the bottom body shell (2) with the stabilization platform (13) by passing through a set of suspension cord receptacle holes (13c, 13d) selected from the plurality of suspension cord receptacle holes (13a-13d).

16. The penile lengthening traction device (10) of claim 1 further comprising a pair of thumb nuts (8a, 8b) mountable on the threaded bolts (7a, 7b) to clamp together the top and bottom clamping assemblies and operationally manipulate or open or close the top clamping assembly in relation to the bottom clamping assembly, thereby allowing to accommodate the penile shaft therebetween.

17. The penile lengthening traction device (10) of claim 1, wherein the top traction pad (5a) and the bottom traction pad (5b) comprises identical tread patterns (5c), and channels (5d) to ensure the top traction pad (5a) and the bottom traction pad (5b) interlock squarely when flipped over to meet face to face.

18. The penile lengthening traction device (10) of claim 1, wherein the top body shell (1) of the top clamping assembly comprises a pair of receptacles (1a,1b) configured for receiving bases (8c, 8d) of the thumb nuts (8a,8b).

19. The penile lengthening traction device (10) of claim 1, wherein one of the pair of receptacles (1a,1b) comprises a bolt hole (1c) to allow the first threaded bolt (7a) to pass therethrough, and one of the pair of receptacles (1a,1b) comprises a slotted bolt hole (1d) to allow the second threaded bolt (7b) to pass therethrough.

20. A penile lengthening traction device (10), comprising:
a top clamping assembly comprising
a top body shell (1),
a top body cover (3a),
a pair of top contact pads (4a, 4b), and
a top traction pad (5a);
a bottom clamping assembly
a bottom body shell (2),
a bottom cover (3b),
a pair of bottom contact pads (4c 4d), and
a bottom traction pad (5b);
wherein, the top clamping assembly and the bottom clamping assembly are operationally coupled using a first threaded bolt (7a), and a second threaded bolt (7b) for hanging appropriate weight thereon;
wherein the top clamping assembly is openable in relation to the bottom clamping assembly to accommodate a penile shaft therebetween; and
wherein the top body shell (1) comprises a first set of holes (9e, 9f) and the bottom body shell (2) comprises a second set of holes (9g,9h) for receiving a first set of suspension cords (9a, 9b), and a second set of suspension cords (9c, 9d) for attachment to a four-cord carabiner attachment assembly (20) having a carabiner attachment hole (12a) for suspending a desired weight thereto.

21. The penile lengthening traction device (10) of claim 19, wherein the four-cord carabiner attachment assembly (20) comprises a stabilization platform (13) with a plurality of suspension cord receptacle holes (13a-13d), a threaded screw attachment knob (13e), and a body (12) with the carabiner attachment hole (12a).
